(19) United States
(12) Patent Application Publication
(10) Pub. No.: US 2022/0015825 A1
(43) Pub. Date: Jan. 20, 2022

(54) SHEATH FOR ABLATION PROBE AND METHODS OF USE THEREOF

(71) Applicant: Vanderbilt University, Nashville, TN (US)

(72) Inventors: William D. MERRYMAN, Nashville, TN (US); Joshua GAFFORD, Nashville, TN (US); Matthew BERSI, Nashville, TN (US); Gregory MICHAUD, Nashville, TN (US); Robert WEBSTER, Nashville, TN (US)

(21) Appl. No.: 17/294,753

(22) PCT Filed: Nov. 20, 2019
(86) PCT No.: PCT/US2019/062360

§ 371 Date: May 18, 2021

Related U.S. Application Data
(60) Provisional application No. 62/769,677, filed on Nov. 20, 2018.

(51) Int. Cl. A61B 18/14

(52) U.S. Cl. CPC A61B 18/1492; A61B 2018/00577

(57) ABSTRACT

One aspect of the present disclosure includes a sheath configured to receive an ablation probe. The sheath includes a temperature-controlled anchor element thereon configured to attach the sheath to bodily tissue.

### RELATED APPLICATIONS

This application claims the benefit of U.S. Provisional Application Ser. No. 62/769,677, filed Nov. 20, 2018, entitled “Cryo Sheath Anchoring for Precision Cardiac Ablation.” This provisional application is hereby incorporated by reference in its entirety for all purposes.

### TECHNICAL FIELD

The present disclosure relates generally to a sheath for receiving an ablation probe and, more particularly, to a sheath for receiving an ablation probe wherein the sheath has a temperature-controlled anchor element.

### BACKGROUND

Cardiac arrhythmias, including atrial fibrillation, are common and dangerous medical conditions, especially in the aging population. A cardiac arrhythmia is any abnormal heart rate or rhythm. In patients with normal sinus rhythm, the heart is electrically excited to beat in a synchronous, patterned fashion. In patients with cardiac arrhythmia, abnormal regions of cardiac tissue do not follow the synchronous beating cycle associated with normally conductive tissue in patients with sinus rhythm. Instead, the abnormal regions of cardiac tissue aberrantly conduct to adjacent tissue, thereby disrupting the cardiac cycle into an asynchronous cardiac rhythm.

Atrial fibrillation in particular is a form of cardiac arrhythmia where there is disorganized electrical conduction in the atria that causes rapid uncoordinated contractions that result in ineffective pumping of blood into the ventricle and a lack of synchrony. During atrial fibrillation, the atrioventricular node receives electrical impulses from numerous locations throughout the atria instead of only from the sinus node. This causes the atrioventricular node to produce an irregular and rapid heartbeat. As a result, blood may pool in the atria increasing the risk for blood clot formation. The major risk factors for atrial fibrillation include age, coronary artery disease, rheumatic heart disease, hypertension, diabetes, and thyrotoxicosis.

Radiofrequency (RF) ablation is an effective therapy for treating atrial and ventricular rhythm disturbances. RF ablation is a procedure that uses radiofrequency energy to destroy a small area of heart tissue that is causing rapid and irregular heartbeats. Destroying this tissue helps restore the heart's regular rhythm. RF ablation targets the key elements of reentrant pathways and/or abnormal ectopic loci without damaging significant amounts of adjacent healthy myocardium and coronary vessels.

### SUMMARY

In one aspect, the present disclosure can include a sheath for receiving an ablation probe, the sheath comprising a sheath body comprising a first outer wall surface and a first inner wall surface, wherein the first outer wall surface and the first inner wall surface extend between a proximal end and a distal end of the sheath body; one or more peripheral lumens located between the first outer wall surface and the first inner wall surface, wherein the one or more peripheral lumens extend between the proximal end and distal end of the sheath body; and a central lumen that extends between the proximal end and the distal end of the sheath body and is defined by the first inner wall surface; and a temperature-controlled anchor element operably coupled to the distal end of the sheath body, the anchor element comprising a proximal end, a distal end, a second outer wall surface and a second inner wall surface extending between the proximal and distal ends of the anchor element, and a second central lumen extending between the proximal and distal ends of the anchor element, wherein the second central lumen is defined by the second inner wall surface and is in fluid communication with the first central lumen.

In another aspect, the present disclosure can include a method for ablating a target bodily tissue, the method comprising delivering a sheath to a target bodily tissue, wherein the sheath comprises a sheath body comprising a first central lumen and a temperature-controlled anchor element comprising a second central lumen that is in fluid communication with the first central lumen; cooling the anchor element to a temperature sufficient to attach the sheath to the target bodily tissue; and activating a radio frequency (RF) ablation probe, being housed within the first and second central lumens of the sheath, to ablate the target bodily tissue.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1A is a perspective view of a sheath constructed in accordance with one aspect of the present disclosure;

FIG. 1B is a perspective view of a sheath comprising a sheath body and a temperature controlled anchor element constructed in accordance with one aspect of the present disclosure;

FIG. 2A is a perspective view of a sheath constructed in accordance with another aspect of the present disclosure;

FIG. 2B is a magnified cut-away view of the sheath in FIG. 2A;

FIG. 3A is a cross-sectional view of the sheath body in FIG. 1B wherein the sheath body comprises a peripheral lumen adapted to convey a coolant therethrough and a vacuum channel;

FIG. 3B is a cross-sectional view of the temperature controlled anchor element in FIG. 1B in accordance with one aspect of the present disclosure;

FIG. 4A is a cross-sectional view of the sheath body in FIG. 1B wherein the sheath body comprises a peripheral lumen adapted to convey a coolant therethrough, a vacuum channel, and a peripheral lumen configured to receive a thermocouple wire;

FIG. 4B is a cross-sectional view of the temperature controlled anchor element in FIG. 1B in accordance with another aspect of the present disclosure;

FIG. 5 is a perspective view of a sheath configured to house a steerable introducer and an ablation probe;

FIG. 6 is process flow diagram illustrating a method for ablating tissue according to an aspect of the present disclosure.

### DETAILED DESCRIPTION

### I. Definitions

Unless otherwise defined, all technical terms used herein have the same meaning as commonly understood by one of ordinary skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

All numeric values are herein assumed to be modified by the term “about,” whether or not explicitly indicated. The term “about” generally refers to a range of numbers that one of skill in the art would consider equivalent to the recited value (i.e., having the same function or result). In some instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the apparatus in use or operation in addition to the orientation depicted in the figures. For example, if the apparatus in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

As used herein, the term “subject” can refer to any warm-blooded organism including, but not limited to, human beings, pigs, rats, mice, dogs, goats, sheep, horses, monkeys, apes, rabbits, cattle, etc.

As used herein, the terms “body tissue” or “bodily tissue” can refer to any biological tissue, such as organs, tendons, muscle, bone, skin, etc. In one example, the terms “body tissue” or “bodily tissue” can include cardiac tissue. Cardiac tissue can include, in some instances, epicardium, myocardium, endocardium, or a portion thereof, and heart atrium tissue.

As used herein, the term “ablate” can refer to the removal or destruction of target body tissue. In the specific instance of cardiac ablation, the term “ablate” can refer to the scaring of small areas in the heart that may be involved in an abnormal heart rhythm. The scarring can prevent abnormal electrical signals or rhythms from moving through the heart. The term “substantially ablate” can mean removal or destruction of at least 50% of the target body tissue, removal or destruction of about 50-60% of the target body tissue, removal or destruction of about 60-70% of the target body tissue, removal or destruction of about 70-80% of the target body tissue, removal or destruction of about 80-90% of the target body tissue, or removal or destruction of about 90-99% of the target body tissue.

As used herein, the term “in fluid communication” can refer to a communication between two sections, components, or features of the systems of the present disclosure. In some instances, this communication may be a direct connection or a direct path between two sections, components, or features or, alternatively, may include one or more intervening sections in the path between two sections, components, or features of the systems of the present disclosure.

### II. Overview

The present disclosure relates generally to a sheath for receiving an ablation probe, and more particularly, to a sheath for receiving an ablation probe wherein the sheath has a temperature-controlled anchor element, and related methods of use.

The present disclosure seeks to overcome challenges associated with RF ablation therapy. When carrying out an RF ablation procedure a physician needs to deliver a consistent RF ablation to cardiac tissue for periods of up to two minutes. While delivering the RF ablation, the tip of the RF probe must remain stable and motionless. This is a current challenge and limitation of RF ablation. It is particularly difficult to treat arrhythmias that are located in the right atrium of the heart or within the myocardial wall that undergo significant motion with each cardiac cycle. The present disclosure seeks to overcome this limitation by providing a temperature controlled sheath that can be precisely anchored to a tissue region of interest. Once the sheath is anchored to the bodily tissue, a physician can use an RF ablation probe to deliver RF energy to the specific tissue of interest and ablate the tissue.

### III. Sheath

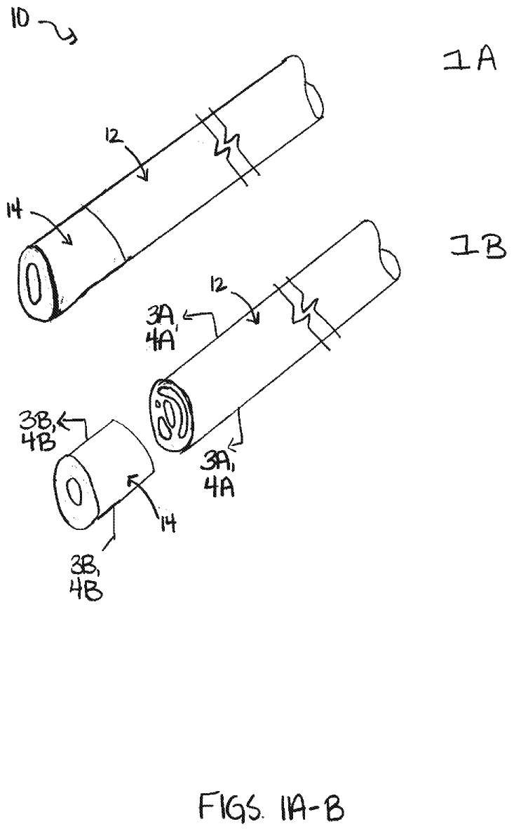

One aspect of the present disclosure is shown in FIGS. 1A-B and includes a sheath 10 for receiving an ablation probe. The sheath 10 can comprise a sheath body 12 and a temperature-controlled anchor element 14 that is operably coupled to the sheath body 12.

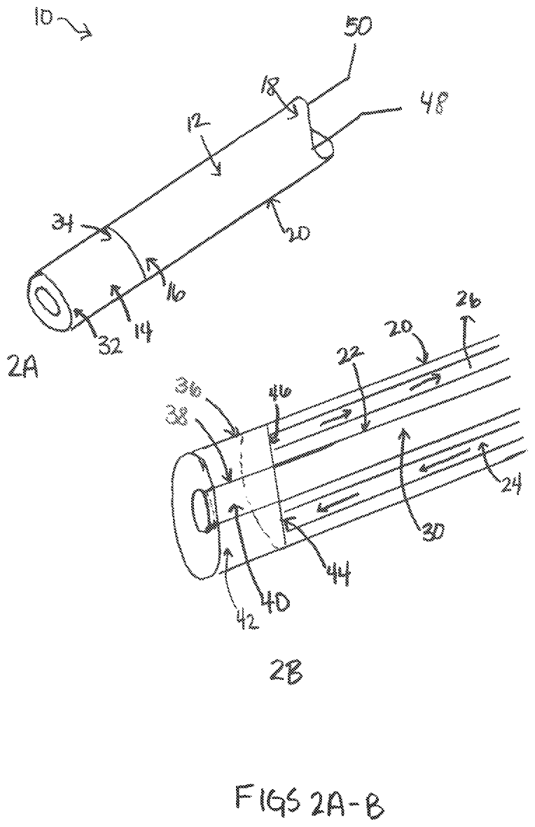

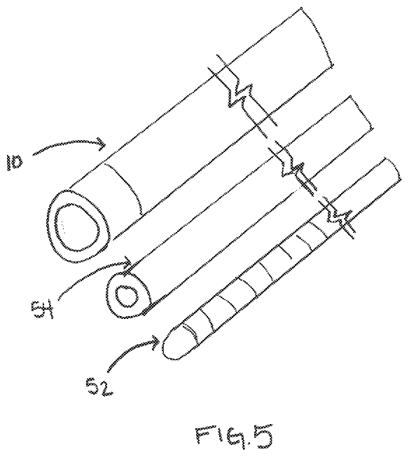

In another aspect, the sheath 10 can be configured as shown in FIGS. 2A-B. In some instances, the sheath body 12 has an elongated, generally cylindrical configuration with a distal end 16 oppositely disposed from a proximal end 18. The sheath body 12 can include a first outer wall surface 20 and an oppositely disposed first inner wall surface 22 (FIG. 2B). One or more peripheral lumens can be located between the first outer wall surface 20 and the first inner wall surface 22 and can extend between the proximal end 18 and distal end 16 of the sheath body 12. In one instance, a first peripheral lumen 24 can be adapted to convey a coolant therethrough. In another instance, a second peripheral lumen 26 can be a vacuum channel. In a further instance, a third peripheral lumen 28 can house a thermocouple wire (FIG. 4A). The sheath body 12 can also include a first central lumen 30 that extends between the proximal end 18 and the distal end 16 of the sheath body 12. The first central lumen 30 can be defined by the first inner wall surface 22.

The outside diameter of the sheath body 12 can vary depending upon the intended application of the sheath 10. In some instances, the outside diameter of the sheath body 12 can be about 12 Fr to about 15 Fr or more. In one example, the outside diameter of the sheath body 12 can be about 12.5 Fr.

The diameter of central lumen 30 can vary depending on the intended application of sheath 10. In some instances the diameter of central lumen 30 can be about 9 F to about 12 F. In one example, the diameter of central lumen 30 can be about 9 F.

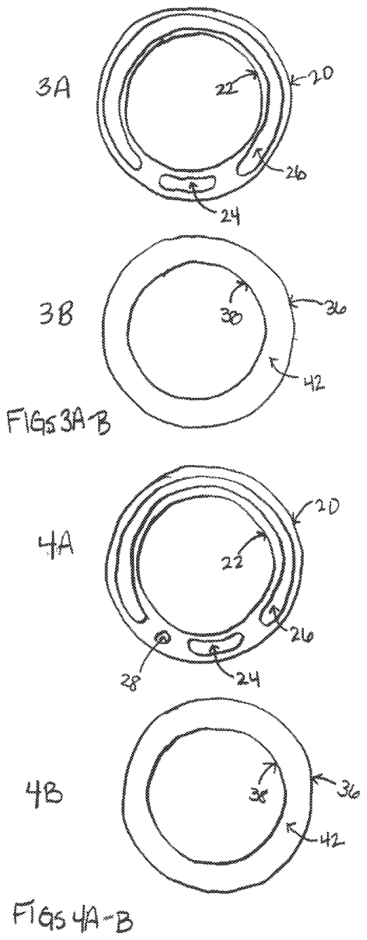

As seen in FIGS. 3A and 4A, where the first peripheral lumen 24 is adapted to receive a coolant therethrough and the second peripheral lumen 26 is a vacuum channel, the cross-sectional area of lumen 26 can be greater than the cross-sectional area of lumen 24. In certain instances, the cross-sectional area of lumen 26 can be at least two, three, four, five, or six times as large as the cross-sectional area of lumen 24.

The sheath body 12 can have a rigid, semi-rigid, or flexible configuration depending upon its intended application. In some instances, the sheath body 12 can be made of one or more biocompatible materials, for example, a polyether block amide (PEBA) (e.g., PEBAX®), polyether ether ketone (PEEK), polyethylene, or polyurethane. In some instances a low friction coating, such as polytetrafluoroethylene (PTFE), can be applied to one or more components of sheath body 12. The material(s) used to form the sheath body 12 can impart sheath 10 with sufficient strength to prevent channel collapse when a vacuum is drawn while maintaining the flexibility required to maneuver the sheath 10 through the vascular system of a subject. In some instances, different portions or regions of the sheath body 12 can be made of different materials to impart each of the portions or regions with a desired flexibility.

In a further aspect, a temperature-controlled anchor element 14 can be operably coupled to the distal end portion 16 of the sheath body 12. In certain instances, the anchor element 14 can be directly coupled to the sheath body 12. The anchor element 14 can include a distal end portion 32 oppositely disposed from a proximal end portion 34. In certain instances, the proximal end portion 34 of the anchor element 14 can be operably coupled to the distal end portion 16 of the sheath body 12. In certain instances, the proximal end portion 34 of the anchor element 14 can be directly coupled to the distal end portion 16 of the sheath body 12.

The anchor element 14 can also include a second outer wall surface 36 and a second inner wall surface 38 extending between the proximal and distal ends of the anchor element 14. The anchor element 14 can further include a second central lumen 40 that extends between the proximal and distal ends of the anchor element 14. The second central lumen 40 can be defined by the second inner wall surface 38. The second central lumen 40 can be in fluid communication with the first central lumen 30.

The anchor element 14 can be made of one or more biocompatible materials (e.g., copper, stainless steel, or the like) capable of providing a cryogenically-cooled surface. The anchor element 14 can be sized and configured depending upon the intended application of the sheath 10. In one example, the anchor element 14 can comprise a dome-shaped distal tip. The outside diameter of the anchor element 14 can vary depending upon the intended application of the sheath 10. In some instances, the outside diameter of the anchor element 14 can be about 12 F to about 15 F or more. In one example, the outside diameter of the anchor element 14 can be about 12.5 F. In one instance, the outside diameter of the anchor element 14 can be the same as the outside diameter of the sheath body 12. In another instance, the outside diameter of the anchor element 14 can differ from the outside diameter of the sheath body 12.

The diameter of second central lumen 40 can vary depending on the intended application of sheath 10. In some instances, the diameter of second central lumen 40 can be about 9 F to about 12 F. In one example, the diameter of the second central lumen 40 can be about 9 F. In a further instance, the diameter of the second central lumen 40 can be the same as the diameter of the first central lumen 30. In another instance, the diameter of the second central lumen 40 can differ from the diameter of the first central lumen 30.

The anchor element 14 can further include an expansion chamber 42 located between the second outer wall surface 36 and the second inner wall surface 38 (FIGS. 2B, 3B, 4B). The expansion chamber 42 can be in fluid communication with one or more of the peripheral lumens. For example, the expansion chamber 42 can be in fluid communication with the first peripheral lumen 24 and with the second peripheral lumen 26.

Sheath 10 can be attached to bodily tissue through anchor element 14. More specifically, anchor element 14 can be attached to bodily tissue by adhering the anchor element 14 to the bodily tissue. The surface of anchor element 14 can be sufficiently cooled so that when it comes into contact with the bodily tissue it forms a congealed adherence layer, such as a solid ice or frozen layer that is formed from tissue and/or fluids adjacent the tissue. In some instances, the anchor element 14 can be configured to provide a cryogenically-cooled surface at a temperature that is higher than the temperature used to cryogenically ablate bodily tissue, but sufficiently cool so as to form a frozen or congealed adherence layer to anchor the sheath 10 to the bodily tissue. In certain instances the anchor element 14 can be configured to be cooled to a temperature ranging from about −80° C. to −30° C.

The anchor element 14 can be cooled to a temperature ranging from about −80° C. to −30° C. by, for example, conveying a coolant through the first peripheral lumen 24 into the expansion chamber 42 and applying a vacuum to the expansion chamber 42 through the second peripheral lumen 26. By circulating the coolant in this manner the anchor element 14 can provide a cryogenically-cooled surface.

Referring to FIG. 2B, each of the first and second peripheral lumens 24 and 26 can include an open distal end 44 and 46 respectively, which are in fluid communication with the expansion chamber 42 so that a coolant can flow therethrough. In some instances, the first peripheral lumen 24 is configured to flow a coolant from a coolant source 48 (FIG. 2A) into the expansion chamber 42 (FIG. 2B). In such instances, a proximal end (not shown) of the first peripheral lumen 24 can be fluidly connected to the coolant source 48. In certain instances, one or more connectors and/or adaptors can be used to connect the first peripheral lumen 24 to the coolant source 48. In other instances, the second peripheral lumen 26 (FIG. 2B) can be configured to flow the cooled fluid from the expansion chamber 42 through the second peripheral lumen 26 towards a vacuum source 50 (FIG. 2A). In such instances, a proximal end (not shown) of the second peripheral lumen 26 can be fluidly connected to the vacuum source 50. In certain instances, one or more connectors and/or adaptors can be used to connect the second peripheral lumen 26 to the vacuum source 50.

The coolant source 48 can include any vessel or container capable of serving as a reservoir for the coolant. In one example, the coolant source 48 can comprise a handheld canister (not shown). The coolant source 48 can be operated manually or automatically. The coolant can be, for example, liquid nitrogen or nitrous oxide gas.

The vacuum source 50 can include any suitable pump or similar device capable of manually or automatically providing a vacuum to flow a cryogenically-cooled fluid from the expansion chamber 42 through the second peripheral lumen 26.

In another aspect, a controller (not pictured) can be configured to control one or more components of the sheath 10. In some instances, the controller can include an anchor control module (not shown) configured to control the anchor element 14. For example, the amount of the coolant and/or the temperature of the anchor element 14 can be controlled by the anchor control module. In another instance, the controller can include an ON/OFF toggle switch that can control the flow of coolant and the pull of the vacuum. Thus, the controller can be in electrical communication with the coolant source 48 and the vacuum source 50.

In some instances, the controller can include circuitry (e.g., a microprocessor, memory, etc.) and software (e.g., one or more algorithms) in electrical communication with the component(s) of the sheath 10. In some instances, the controller (e.g., the software) can pre-programmed to selectively control the temperature of the anchor element 14. The controller can be powered by a power source (not shown), such as a battery.

As shown in FIG. 5, the sheath 10 can be configured to receive and house an ablation probe 52 and/or a steerable introducer 54. In one aspect, each of the first and second central lumens 30 and 40 can have a cross-sectional area sufficiently large to house a steerable introducer. In another instance, each of the first and second central lumens 30 and 40 can have a cross-sectional area sufficiently large to house an ablation probe, such as an RF ablation probe. In a further instance, each of the first and second central lumens 30 and 40 can have a cross-sectional area sufficiently large to house a steerable introducer and an ablation probe. The steerable introducer may be, e.g., an Agilis Steerable Introducer or a Fustar Steerable Introducer. The sheath 10 can be used with a variety of known ablation probes and with any known RF catheters.

### IV. Methods

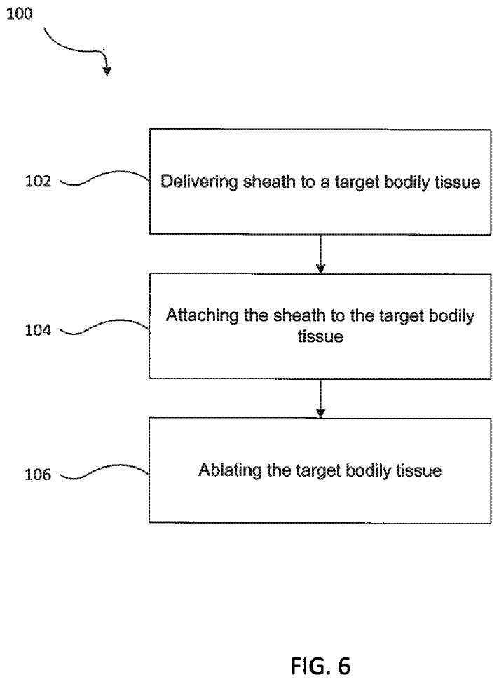

Another aspect of the present disclosure is illustrated in FIG. 6 and includes a method 100 for ablating bodily tissue in a subject. The method is useful for any percutaneous procedure where anchored ablation would provide more precise spatial control and a greater degree of stability. For example, the method 100 can find use in treatments that require the use of RF ablation in dynamic environments, such as the treatment of cardiac arrhythmias where there is moving target tissue. Although the method is directed to ablating tissue, it will be appreciated that the method may generally find use in other cardiac procedures involving a catheter that require prolonged anchoring and stability including, for example, injections and biopsies.

At step 102, a sheath 10 is introduced into the vasculature of the subject, using a known percutaneous surgical technique, and is delivered to a target bodily tissue. For example, the sheath 10 can be delivered to bodily tissue in the heart. In certain instances, the sheath 10 can be delivered to the target bodily tissue using a steerable introducer housed within the first and second central lumens of the sheath 10. The steerable introducer may be, e.g., an Agilis or Fustar Steerable Introducer.

At step 104, the anchor element 14 of sheath 10 can be attached to the target bodily tissue. In certain instances, the target bodily tissue can be bodily tissue in the right atrium of the heart. In other instances, the target bodily tissue can be the myocardial wall of the heart. The anchor element 14 of the sheath 10 can be attached to the target bodily tissue while the target bodily tissue is moving with respect to the surrounding cardiac tissue.

Either before or while contacting the target bodily tissue with the anchor element 14, the anchor element 14 can be cooled in order to attach the sheath 10 to the target bodily tissue. In one example, the anchor element 14 can be cooled to a temperature between about −80° C. and about −30° C. The cooled surface of the anchor element 14 can attach, e.g., freeze the sheath 10 to the target bodily tissue.

At step 106, an ablation probe housed within the first and second central lumens of the sheath 10 can ablate the target bodily tissue. The ablation probe can be a RF ablation probe; however, any suitable ablation probe can be used, including microwave energy, laser energy, heat energy and/or a cryogenically-cooled ablation probe. The ablation probe can deliver ablation energy (e.g., RF energy) for a time and at a temperature sufficient to ablate or substantially ablate the target bodily tissue. For example, with regard to atrial fibrillation, the ablation probe can deliver energy sufficient to scar or destroy the heart tissue so as to disrupt the faulty electrical signals causing the arrhythmia. In some instances, the amount of energy (J) delivered to the target bodily tissue can vary depending upon the severity of the dysfunction, the location of the diseased or dysfunctional tissue, the overall health of the subject, etc. For example, a desired amount of power (W), such as about 20 W to about 100 W or more can be delivered to the target bodily tissue for a desired period of time (e.g., about 5 seconds to about 60 seconds or more).

In instances where a steerable introducer is used to deliver the sheath 10 to a target bodily tissue, the ablation probe can be housed within the steerable introducer. In some instances, the ablation probe can be inserted and housed within the steerable introducer after the sheath 10 is attached to the target bodily tissue. In other instances, the ablation probe can be inserted and housed in the steerable introducer before the sheath 10 is attached to the target bodily tissue.

From the above description, those skilled in the art will perceive improvements, changes and modifications. Such improvements, changes, and modifications are within the skill of one in the art and are intended to be covered by the appended claims. All references cited herein and listed above are incorporated by reference in their entireties as needed and as discussed herein.

The following is claimed:

1. A sheath for receiving an ablation probe, the sheath comprising:
a sheath body comprising:
a first outer wall surface and a first inner wall surface, wherein the first outer wall surface and the first inner wall surface extend between a proximal end and a distal end of the sheath body;
one or more peripheral lumens located between the first outer wall surface and the first inner wall surface, wherein the one or more peripheral lumens extend between the proximal end and distal end of the sheath body; and
a central lumen that extends between the proximal end and the distal end of the sheath body and is defined by the first inner wall surface; and
a temperature-controlled anchor element operably coupled to the distal end of the sheath body, the anchor element comprising a proximal end, a distal end, a second outer wall surface and a second inner wall surface extending between the proximal and distal ends of the anchor element, and a second central lumen extending between the proximal and distal ends of the anchor element, wherein the second central lumen is defined by the second inner wall surface and is in fluid communication with the first central lumen.

2. The sheath of claim 1, wherein the temperature-controlled anchor element comprises an expansion chamber located between the second outer wall surface and the second inner wall surface, and the anchor element is in fluid communication with one or more peripheral lumens.

3. The sheath of claim 1, wherein the temperature-controlled anchor element comprises a thermally-conductive material.

4. The sheath of claim 3, wherein the thermally-conductive material is copper.

5. The sheath of claim 1, wherein a first peripheral lumen is adapted to convey a coolant therethrough.

6. The sheath of claim 1, wherein a second peripheral lumen is a vacuum channel.

7. The sheath of claim 1, wherein a first peripheral lumen is adapted to convey a coolant therethrough and a second peripheral lumen is a vacuum channel.

8. The sheath of claim 1, wherein a third peripheral lumen is configured to receive a thermocouple wire.

9. The sheath of claim 7, wherein the cross-sectional area of the second peripheral lumen is greater than the cross-sectional area of the first peripheral lumen.

10. The sheath of claim 9, wherein the cross-sectional area of the second peripheral lumen is at least twice as large as the cross-sectional area of the first peripheral lumen.

11. The sheath of claim 1, wherein the temperature-controlled anchor element is configured to be cooled to a temperature of between about −80° C. and −30° C.

12. The sheath of claim 1, wherein each of the first and second central lumens have a cross-sectional area sufficiently large to receive a steerable introducer.

13. The sheath of claim 12, wherein a diameter of each of the first and second central lumens is between about 9 F and about 12 F.

14. A method for ablating a target bodily tissue, the method comprising:
delivering a sheath to a target bodily tissue, wherein the sheath comprises a sheath body comprising a first central lumen and a temperature-controlled anchor element comprising a second central lumen that is in fluid communication with the first central lumen;
cooling the anchor element to a temperature sufficient to attach the sheath to the target bodily tissue; and
activating a radio frequency (RF) ablation probe, being housed within the first and second central lumens of the sheath, to ablate the target bodily tissue.

15. The method of claim 14, wherein the sheath is delivered to the target bodily tissue using a steerable introducer being housed within the first and second central lumens of the sheath.

16. The method of claim 15, wherein the RF ablation probe is housed within the steerable introducer.

17. The method of claim 16, wherein the RF ablation probe is inserted into and housed within the steerable introducer after the sheath is attached to the target body tissue.

18. The method of claim 16, wherein the RF ablation probe is inserted into and housed within the steerable introducer before the sheath is attached to the target body tissue.

19. The method of claim 16, wherein the target bodily tissue is in the right atrium of the heart.

20. The method of claim 16, wherein the target bodily tissue is myocardial wall bodily tissue.
